(19) United States
(12) Patent Application Publication
(10) Pub. No.: US 2010/0292621 A1
(43) Pub. Date: Nov. 18, 2010

(54) CARPAL/HAND IMMOBILIZER

(76) Inventors: David Thomas, Sayre, PA (US); Linda Thomas, Sayre, PA (US)

Correspondence Address: LAW OFFICE OF JERRY D. HAYNES , P.A, P.O. Box 350392, Fort Lauderdale, FL, 33335, US

(21) Appl. No.: 12/467,210

(22) Filed: May 15, 2009

(51) Int. Cl. A61F 5/00

(52) U.S. Cl. 602/22; 602/21

(57) ABSTRACT

The present invention relates to a hand immobilizing device for placement over the outside of a hand. The hand immobilizing device may include: a pillow brace, where said brace extends from the top knuckles to the wrist of hand; at least one strap securing the pillow brace in place; a slightly curved top portion that causes the knuckles to bend downward; and slightly curved bottom portion that causes the hand to bend upward. In one particular embodiment, the brace extends upwardly to the distal knuckle of the middle finger and covers the small finger. The brace may also extend upwardly to the distal knuckle of the thumb. The straps may utilize Velcro to secure the brace to the hand.

### BACKGROUND OF THE INVENTION

1. Field of Invention

The present invention relates to a carpal/hand immobilizer that treats median neuropathy.

2. Description of Related Art

Many workers suffer from carpal tunnel syndrome due to repetitive tasks such as typing, cutting, use of vibrating hand tools and hand assembly of products are just a few areas prone to cause carpal tunnel syndrome. The carpal tunnel syndrome is a condition wherein the median nerve is compressed within the tunnel and causes pain or numbness of the wrists/hands and/or tingling or pain in the median nerve distribution. These problems occur along the thumb, index, middle and radial half of the ring finger causing possible weakness in the thenar muscles.

Many devices exist which attempt to alleviate the pain and problems associated with carpal tunnel syndrome. Many of these devices involve conventional splints and other plastic or cushion supports. The devices are attached to the hand and the wrist areas. More aggressive means of addressing the problem of carpal tunnel syndrome involve use of medication and possibly surgery. Although some of these methods may have a certain degree of effectiveness, many are ineffective in alleviating and rejuvenating the carpal tunnel area in an effective manner. In addition to pain suffered while performing activities individuals also suffer pain while sleeping and therefore are awakened with throbbing pain in their carpal tunnel areas.

### SUMMARY OF THE INVENTION

The present invention relates to a hand immobilizing device for placement over the outside of a hand comprising: a pillow brace, where said brace extends from the top knuckles to the wrist of hand; at least one strap securing the pillow brace in place; a slightly curved top portion that causes the knuckles to bend downward; and slightly curved bottom portion that causes the hand to bend upward. In one particular embodiment, the brace extends upwardly to the distal knuckle of the middle finger and covers the small finger. The brace may also extend upwardly to the distal knuckle of the thumb. The straps may utilize Velcro to secure the brace to the hand.

### BRIEF DESCRIPTION OF DRAWINGS

FIG. 1A shows an outline of the area addressed by a carpal hand immobilizer according to the present invention.

FIG. 1B depicts the hand immobilizer according to present invention fitted over the outside of a hand.

FIG. 1C shows the palm of the hand where the hand immobilizer is attached to the outside portion thereof.

### DETAILED DESCRIPTION

The present invention provides a hand immobilizer that is attached to the outer portion of the hand and provides a means to regulate wrist flexion, movement and management of carpal tunnel pain. The hand immobilizer extends from just below the tips of the fingers to just above the wrist. The hand immobilizer straps into a fixed position and immobilizes the hand in order to alleviate and rejuvenate the carpals running through the wrists. This immobilizer alleviates tension on the median nerve and therefore reduces pain, numbness and other problems associated with carpal tunnel syndrome.

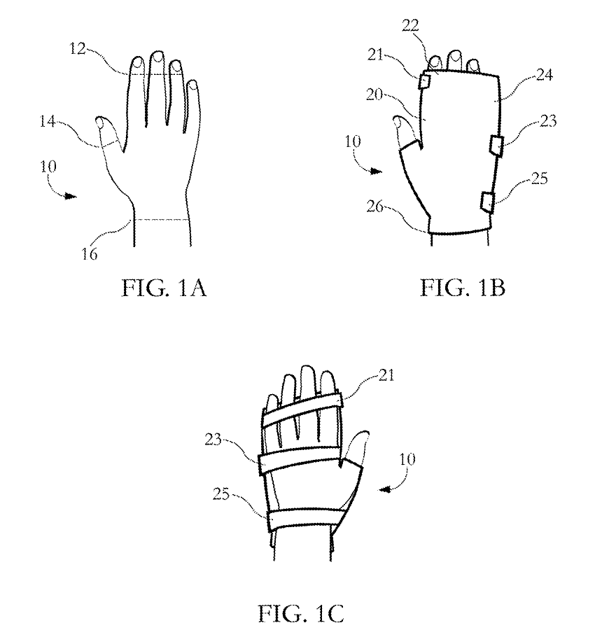

The FIG. 1A depicts a hand 10 and marks the key points associated with the hand immobilizer according to the present invention. A top knuckle line 12 indicates the top placement line for the hand immobilizer. Also shown in FIG. 1A is the top thumb knuckle position and the bottom wrist 16 position. When the hand immobilizer is placed across the hand 10 these are the key boundaries that define the coverage area of the hand immobilizer. FIG. 1B shows the placement of the hand immobilizer 20 over a person's hand. The hand immobilizer includes straps 21, 23, 25 wherein the straps extend across the palm and the inside knuckles of the hand in order to hold the hand immobilizer 20 in place. Placement of the hand immobilizer 20 follows the outline depicted in FIG. 1A, where the top end of the hand immobilizer 20 cuts across the top knuckle position 12 extends to the top of the wrist 16 and to the top thumb knuckle 14. The hand immobilizer 20 is a pillow shaped brace where the top 22 of the hand immobilizer 20 pushes the finger in an upward position and the wrist portion 26 also pushes the hand in an up position. The knuckle position 24 of the hand immobilizer 24 presses the knuckles in a downward position and therefore opens up the median nerves to alleviate tension within the carpal tunnel.

FIG. 1C depicts the palm section of the hand 10 where the straps 21, 23, 25 extend across the palm and attach to the outside of the hand immobilizer 20 to hold the hand immobilizer 20 in place. The straps utilize Velcro in order to hold the straps and the hand immobilizer in place. The Velcro strapping enables the immobilizer to be adjusted and secured in accordance with the user's specifications.

The hand immobilizer 20 holds the fingers in this fixed position as described above and allows the nerves to rejuvenate and alleviates swelling or numbness that may be associated with them. The hand immobilizer 20 includes a plastic support and closure that fits over the mid finger region in order to hold the hand in the position described above where the upper portion is flexed upward and the knuckles are flexed downward. The hand immobilizer 20 keeps the muscles of the hand under control and enables the user to enjoy relief from carpal tunnel related problems.

What is claimed is:

1. A hand immobilizing device for placement over the outside of a hand comprising:
a. a pillow brace, where said brace extends from the top knuckles to the wrist of hand;
b. at least one strap securing the pillow brace in place;
c. a slightly curved top portion that causes the knuckles to bend downward; and
d. slightly curved bottom portion that causes the hand to bend upward.

2. The hand immobilizing device according to claim 1, where the brace extends upwardly to the distal knuckle of the middle finger and covers the small finger.

3. The hand immobilizing device according to claim 1, where the straps utilize Velcro to secure the brace to the hand.

4. The hand immobilizing device according to claim 1, where the brace extends upwardly to the distal knuckle of the thumb.

5. A hand immobilizing device for placement over the outside of a hand comprising:
a. a pillow brace, where said brace extends from the top knuckles to the wrist of hand;
b. three straps securing the pillow brace in place;
c. a slightly curved top portion that causes the knuckles to bend downward; and
d. slightly curved bottom portion that causes the hand to bend upward.

6. The hand immobilizing device according to claim 1, where the brace extends upwardly to the distal knuckle of the middle finger and covers the small finger.

7. The hand immobilizing device according to claim 1, where the straps utilize Velcro to secure the brace to the hand.

8. The hand immobilizing device according to claim 1, where the brace extends upwardly to the distal knuckle of the thumb.
